(19) United States
(12) Patent Application Publication
(10) Pub. No.: US 2022/0384054 A1
(43) Pub. Date: Dec. 1, 2022

(54) TOOL TO QUANTIFY AIRBORNE-DISEASE TRANSMISSION RISK IN A WORKPLACE SETTING

(71) Applicant: One Concern, Inc., Menlo Park, CA (US)

(72) Inventors: Ahmad Wani, Mountain View, CA (US); Nicole Hu, Mountain View, CA (US); Abhineet Gupta, Menlo Park, CA (US); Anand Sampat, Menlo Park, CA (US); Peter Moore, Menlo Park, CA (US); Xi Guo, Menlo Park, CA (US)

(21) Appl. No.: 17/327,975

(22) Filed: May 24, 2021

(51) Int. Cl. G16H 50/80; G06Q 10/06

(52) U.S. Cl. CPC G16H 50/80; G06Q 10/067; G06Q 10/0635; G06Q 40/08

(57) ABSTRACT

Methods, systems, and computer programs are presented for estimating disease-spreading based on facility and behavioral parameters. One method includes an operation for causing presentation of a user interface (UI) for entering facility parameters. The method further includes an operation for calculating the number of infections at the facility for a predetermined time period. The calculation includes setting values for simulation parameters based on the facility parameters, modeling a contact network for people at the facility, and performing a plurality of simulations to determine the number of infections, the plurality of simulations based on the contact network and the facility simulation parameters. Further, the method includes an operation for causing presentation of the number of infections in the UI.

### TECHNICAL FIELD

The subject matter disclosed herein generally relates to methods, systems, and machine-readable storage media for analyzing the spread of disease at the workplace.

### BACKGROUND

As a pandemic is getting under control, one of the key problems for companies is how to reopen facilities safely, and for facilities that are opened during the pandemic, what are the risks involved and how to prepare to minimize those risks.

There are many parameters involved in planning, including infection rates, distancing within a facility that hosts employees, compliance with mask requirements etc., so it is difficult to determine how the pandemic will affect employees at the facilities that are open.

### BRIEF DESCRIPTION OF THE DRAWINGS

Various of the appended drawings merely illustrate example embodiments of the present disclosure and cannot be considered as limiting its scope.

FIG. 1 is a user interface (UI) for selecting a facility type, according to some example embodiments.

FIG. 2 is a UI for selecting a manufacturing building, according to some example embodiments.

FIG. 3 is a UI for entering the building address.

FIGS. 4A-4B show a UI for presenting an estimated infection impact and options to change facility parameters, according to some example embodiments.

FIG. 5 illustrates a Susceptible-Exposed-Infectious-Recovered (SEIR) model for estimating the number of infected and dead individuals caused by the spread of a virus.

FIG. 6 is an example of the spread for a contact network, according to some example embodiments.

FIG. 7 is a sample architecture for implementing embodiments.

FIG. 8 is a table with product parameters and the allowed values, according to some example embodiments.

FIG. 9 is a table with allowed values based on mask compliance and building layout, according to some example embodiments.

FIG. 10 is a table with allowed values based on social-distance compliance and building layout, according to some example embodiments.

FIG. 11 illustrates some of the metrics for verifying the model, according to some example embodiments.

FIG. 12 is a flowchart of a method for interacting with the planning tool, according to some example embodiments.

FIG. 13 is a flowchart of a method for estimating disease-spreading based on facility and behavioral parameters, according to some example embodiments.

FIG. 14 is a block diagram illustrating an example of a machine upon or by which one or more example process embodiments described herein may be implemented or controlled.

### DETAILED DESCRIPTION

Example methods, systems, and computer programs are directed to estimating disease-spreading based on facility and behavioral parameters. Examples merely typify possible variations. Unless explicitly stated otherwise, components and functions are optional and may be combined or subdivided, and operations may vary in sequence or be combined or subdivided. In the following description, for purposes of explanation, numerous specific details are set forth to provide a thorough understanding of example embodiments. It will be evident to one skilled in the art, however, that the present subject matter may be practiced without these specific details.

What is needed are tools that analyze the current state of the pandemic, the facilities where the employees work, and behavioral parameters to assess risk and plan for a safe working environment.

In one aspect, a tool is presented for assisting employers to understand the effects of a contagious disease in a facility where employees work. The tool provides options to enter the characteristics of the workplace and generates assessments to predict the spread of the disease. Further, the tool allows the user to modify the input parameters, as well as default parameters, and allow the user to perform what-if analysis.

Although embodiments are presented with reference to a highly contagious pandemic such as COVID-19, the same principles may be utilized for other types of contagious diseases, such as the flu, the common cold, Ebola, etc.

One general aspect includes a method that includes an operation for causing presentation of a user interface (UI) for entering facility parameters. The method further includes an operation for calculating the number of infections at the facility for a predetermined time period. The calculation includes setting values for simulation parameters based on the facility parameters, modeling a contact network for people at the facility, and performing a plurality of simulations to determine the number of infections, the plurality of simulations based on the contact network and the facility simulation parameters. Further, the method includes an operation for causing presentation of the number of infections in the UI.

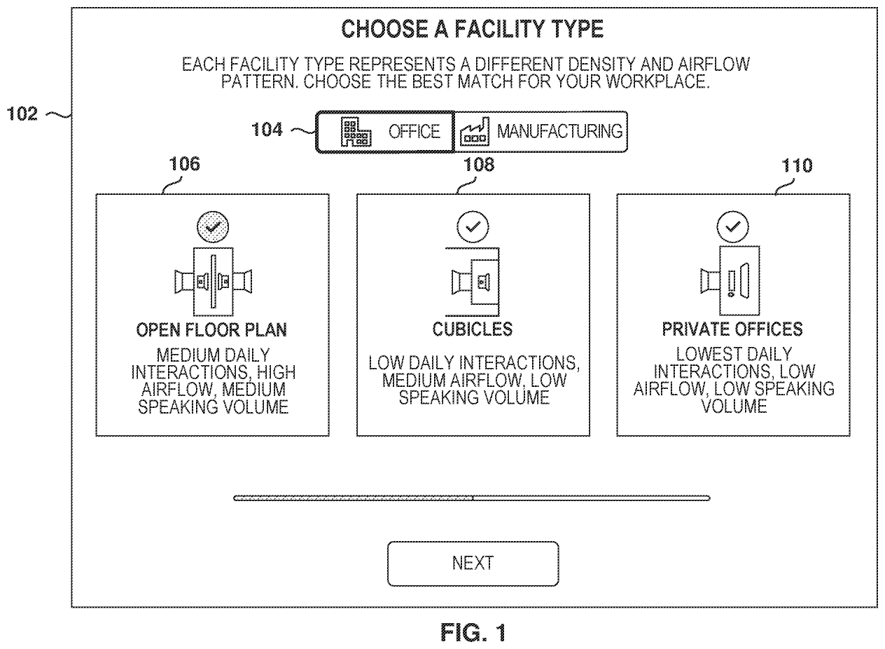

FIG. 1 is a user interface (UI) 102 for selecting a facility type, according to some example embodiments. The tool for analyzing the impact of a contagious disease is referred to herein as the planning tool, which in the case of COVID-19, the tool is called COVID-19 planning tool or COVID Calculator.

The UI 102 of the planning tool is the initial interface for selecting the type of facility under consideration. In some example embodiments, the facilities are divided into two groups: office and manufacturing. The selection menu 104 enables the user to select one of the two types of facilities. Depending on the type of facility selected, additional options for categorizing the facility are provided below.

In the example illustrated in FIG. 1, the Office option has been selected and the additional options include open-floor plan 106, cubicles 108, and private offices 110. “Open-floor plan” is for open spaces without walls separating the workers, such as an open space where multiple workers have desks to perform their work. “Cubicles” refers to an area with a cubicle for each worker (although, in some cases, some workers may share a cubicle), and “Private offices” refers to a space where workers have their own offices with walls and a door (although, in some cases, some workers may share an office).

Once the user makes the appropriate selections, the user may continue with the configuration by selecting the Next button.

In general, the open-floor plan is characterized by medium daily interactions, high airflow, and medium speaking activity. The cubicles are characterized by lower daily interactions, medium airflow, and lower speaking activity. Further, the private offices are characterized by the lowest daily interactions from the three categories, low airflow, and low speaking activity.

Based upon the user's choices, the planning tool utilizes default assumptions about interactive dynamics between people in the space, and these assumptions are used for modeling the spread of the disease, as described in more detail below.

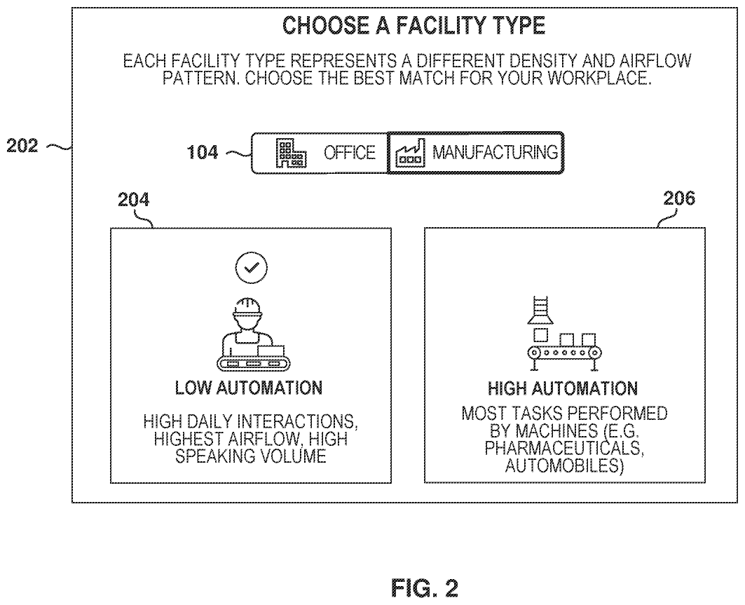

FIG. 2 is a UI 202 for selecting a manufacturing building, according to some example embodiments. The UI 202 shows the options when the user selects the manufacturing option. In some example embodiments, the options associated with manufacturing include low automation 204 and high automation 206.

“Low automation” refers to small- to medium-size manufacturing facilities, and “High automation” refers to large manufacturing facilities. The low-automation option is characterized by high daily interactions, highest level airflow, and the highest speaking volume. Further, the high automation option is for facilities where most tasks are performed by machines, e.g., pharmaceutical manufacturing and automobile manufacturing.

It is noted that the embodiments illustrated in FIGS. 1-2 are examples and do not describe every possible embodiment. Other embodiments may utilize a different number of options, additional options, fewer options, etc. The embodiments illustrated in FIGS. 1-2 should therefore not be interpreted to be exclusive or limiting, but rather illustrative.

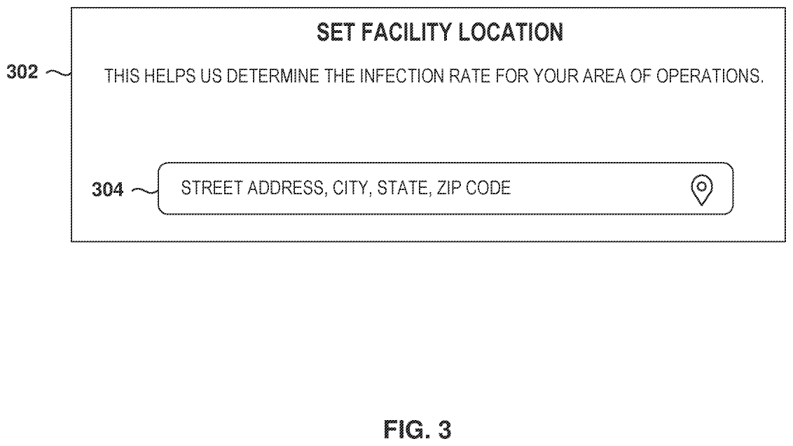

FIG. 3 is a UI 302 for entering the building address. After selecting the facility, the UI 302 provides an entry box 304 for inputting the address of the facility. The address assists in selecting parameters for the facility, such as the current number of cases in the area, the number of new cases per period (e.g., spread rate), number of deaths, etc.

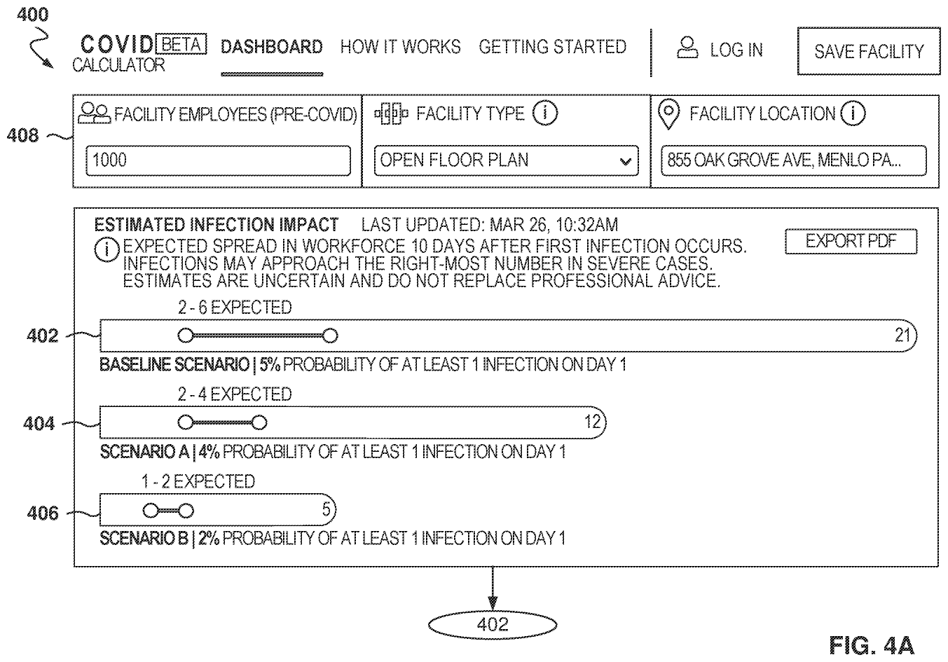

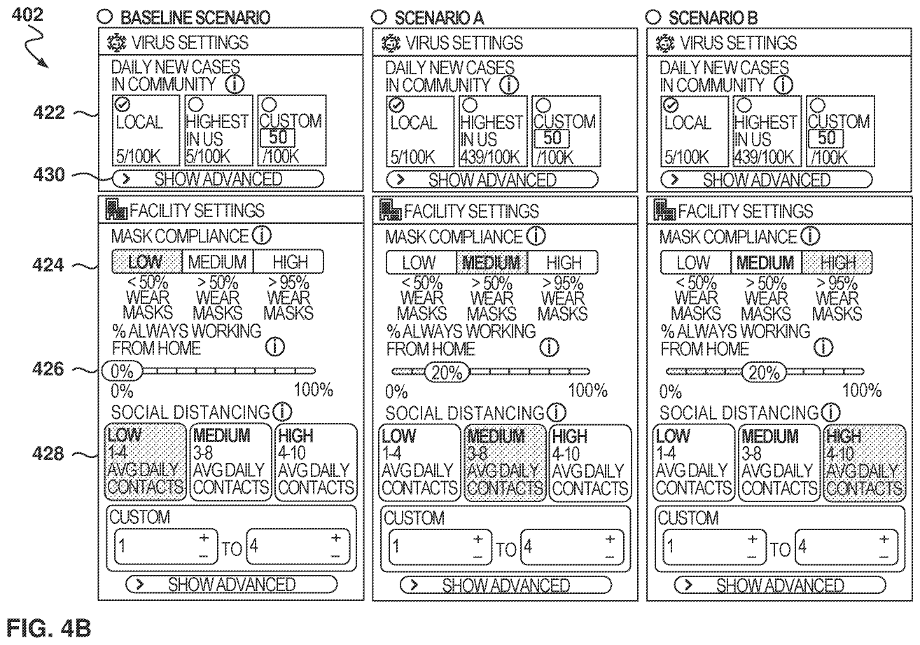

FIGS. 4A-4B show a UI 400 for presenting an estimated infection impact and options to change facility parameters, according to some example embodiments. The UI 400 is presented in FIGS. 4A and 4B, with the top of the UI 400 presented in FIG. 4A and the bottom in FIG. 4B.

After the user enters the parameters for the planning tool, the UI 400 shows initial estimates for the estimated infection impact, which includes one or more scenarios. The estimated infection impact shows the expected spread in the workforce 10 days after the first infection occurs.

In the illustrated example, the scenarios include a baseline scenario 402 with a 5% probability of at least one infection on day 1, a scenario A 404 with a 4% probability of at least one infection on day 1, and a scenario B 406 with a 2% probability of at least one infection on day 1. For each scenario, a graphical bar is presented with a size related to the highest number of cases for severe outbreaks. In the illustrated examples, the high-case marks are 21, 12, and 5.

Further, for each scenario, an expected range is provided which is also presented to scale within each scenario (e.g., expected 2-6 for scenario A 404).

On top of the UI 400, a parameters window 408 includes the selected options, that is, number of facility employees, facility type, and the facility location. The UI 400 provides entry fields for changing the facility parameters to recalculate the estimated infection impact.

FIG. 4B shows the information that characterizes each scenario and provides options for changing the scenarios. In some example embodiments, each scenario includes parameters for daily new cases in the community 422, mask compliance 424 in the facility, percentage of workers working from home 426, and social distancing 428 in the facility.

For the daily new cases in the community 422, three options are presented: local rate, highest rate in the US, and a custom value expressed as number of new cases per 100,000 people. A button is presented for showing additional advanced options.

For the mask compliance 424 in the facility, the user may select low, medium, or high. In some example embodiments, low is when less than 50% of workers wear masks, medium low is when mask compliance is between 50% and 95%, and high is when compliance is greater than 95%.

For the percentage of workers working from home 426, a slide bar is provided where the user can select a value between 0% and 100%.

For the social distancing 428 in the facility, the options are low, medium, and high. Low is for average daily contacts between 1 and 4, medium is between 3 and 8, and high is 4 to 10, although these ranges may be configurable based on the selected facility (e.g., open office vs cubicles). Additionally, the user can enter a custom range by providing the low and high numbers for the range. Additionally, an option is provided to select an advanced option for entering range parameters.

By configuring the parameters for the different scenarios, the manager may compare the consequences of changing policy, such as the number of employees, number of people working from home, mask compliance, social interactions, speaking activity, virus variant, etc. The planning tool allows the user to manage risk and assess the impact of different measures to control the spread of the disease.

Further, the information provided may be used by insurance companies, or by the employer working with the insurance company in order to assess risk and the associated insurance premium. For example, a company may lower the insurance cost by mandating that all employees wear masks, or by reducing the number of employees at the facility to increase social distancing.

Managers may also assess the risk to the business and to their employees, and assess the cost of potential improvements (e.g., adding partitions within an open area) against the risk.

When the user selects the show-advanced option 430, additional features for configuring the scenario are presented. These options include the ability to select a SARS-CoV-2 variant to model, and to select a level of virus transmissibility. The levels of virus transmissibility may be set to the baseline COVID transmissibility, to the transmissibility of newer COVID variants, or to the transmissibility of potential future variants that have not yet occurred but that the user may configure. With these advance options, users can compare outbreak risk under different infection conditions, including hypothetical future scenarios.

It is noted that the embodiments illustrated in FIGS. 4A-4B are examples and do not describe every possible embodiment. Other embodiments may utilize different parameters, a different number of scenarios, different data entry mechanisms, etc. The embodiments illustrated in FIGS. 4A-4B should therefore not be interpreted to be exclusive or limiting, but rather illustrative.

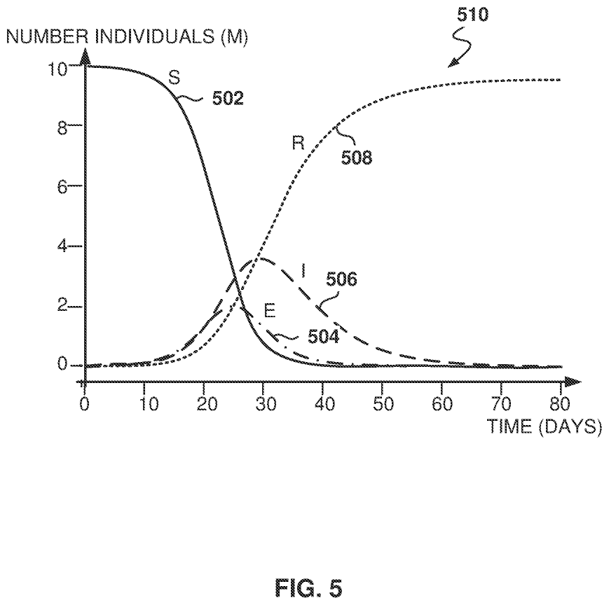

FIG. 5 illustrates a Susceptible-Exposed-Infectious-Recovered (SEIR) model for estimating the number of infected and dead individuals caused by the spread of a virus. The SEIR model has mainly four components: Susceptible (S) 502, Exposed (E) 504, Infectious (I) 506, and Removed (R) 508. S is the fraction of susceptible individuals (i.e., people able to contract the disease), E is the fraction of exposed individuals (i.e., those who have been infected but are not yet infectious), I is the fraction of infective individuals (i.e., those capable of transmitting the disease), and R is the fraction of recovered or removed individuals (i.e., those who have become immune or died).

Chart 510 shows an example evolution of the pandemic, where S 502 begins with the complete population and decays over time, R 508 begins at 0 and grows as people recover, E 504 grows first as more people are exposed and later declines as people recover, and I 506 grows as people get infected and later declines as people recover or die.

Traditional SEIR models are not suitable for modeling short-term spread within facilities, as the traditional models focus on large-scale projections, such as state or country. In some example embodiments, the prediction models used for facilities take into consideration short populations within the facility, the inherent randomness in transition, and focus on a shorter time window than traditional models, e.g., a 10-day time window, although other time windows are also possible.

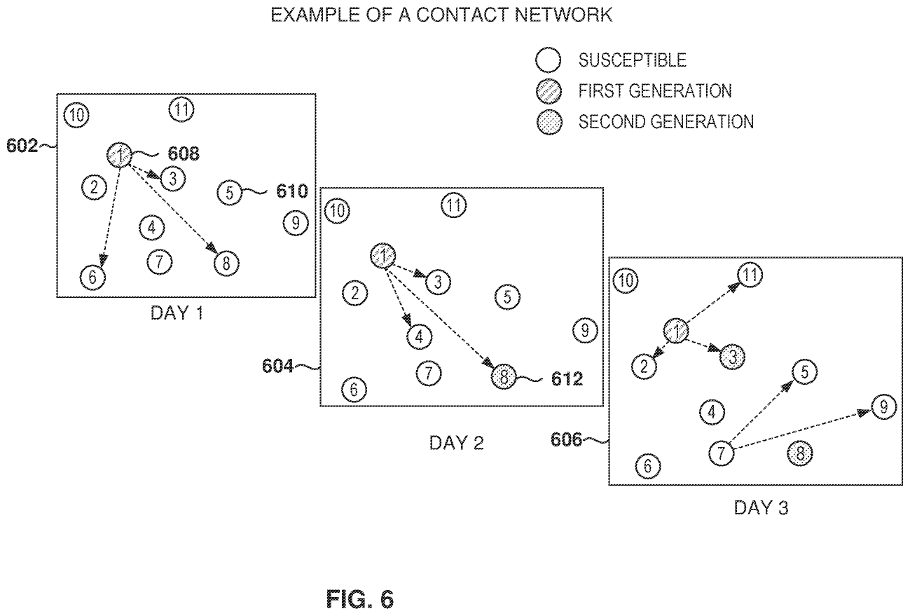

FIG. 6 is an example of the spread for a contact network 602, according to some example embodiments. Contact networks have been used in epidemiological modeling and provide a robust way to consider the individual nature of disease transmission. Contact networks are used to model the transmissions in human populations due to either social contacts (airborne infections) or sexual contacts (sexually transmitted infections).

In some embodiments, two individuals are linked if they have sufficient contact to allow the infection to pass between them, and tracing information for employees is used to build the contact network 602.

There are different types of contact networks, such as random, small world, scale-fee, etc. In some example embodiments, a simplified dynamic random contact network is used to simulate the social interactions inside workplaces, but other types of contact networks may be used.

Often, network epidemiology is based on static contact networks, which are graphs where nodes and edges remain fixed for the duration of the epidemic in the population, and static contact networks assume that contacts are permanent and provide a useful tool for predicting the spread of disease through relatively stable populations. However, social interactions are often quite fluid as new connections form while others dissolve, which provides transient opportunities for disease transmission. A dynamic representation of a contact network, in which nodes and edges shift according to changes in the population, is more appropriate for analyzing facility interactions.

The contact network 602 for day one includes an infected individual I 608 and a plurality of susceptible individuals 610. In day one, the infected individual 1 comes in contact with individuals 3, 6, and 8. For each contact, there is a probability of transmission, and in this example, there is no disease transmission on day one.

On day two (contact network 604), that infected individual 1 comes in contact with individuals 3, 4, and 8, and there is successful transmission to individual 8, which is now part of the second generation 612 of infected individuals.

In some example embodiments, third-generation cases are not considered, but other embodiments may consider a third generation and other generations. Thus, on day 3 (contact network 606), individual 7 is infected and has constant contact with individuals 5 and 9, but without transmission. Further, on day 3, infected individual 1 infects individual 3.

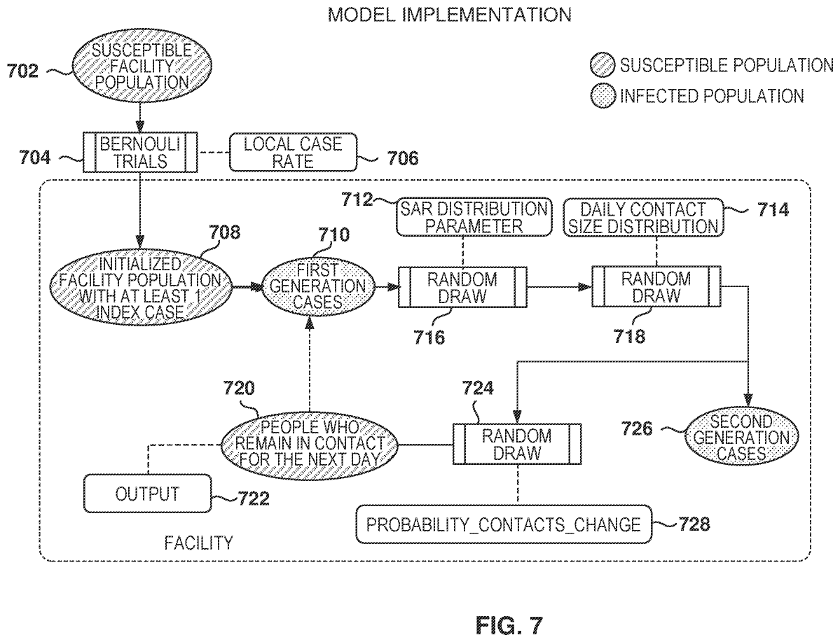

FIG. 7 is a sample architecture for implementing embodiments. In some example embodiments, the model for transmission includes several assumptions. First, the model tracks the first and second generation of infections in the transmission chain, which includes primary cases and secondary cases in a single transmission chain. Second, the period between getting exposed and becoming infectious (referred to as latency period or incubation period) is not modeled. Third, it is assumed that infected individuals will not be identified during the ten-day period; thus, no infected individuals are to be removed from the facility during this time. Further yet, it is assumed that the transmissibility of the index case remains constant throughout the period in the facility. In reality, during the course of infection, the transmissibility depends on the volume of shed viruses, which peaks around 3 days after infection.

The initialized facility population 708 are introduced from the susceptible facility population 702 using a Bernoulli trial 704 with the local case rate 706 used as the success rate of infection. These are the first-generation cases. A Bernoulli trial is a random experiment with two possible outcomes: success and failure, and the probability of success is the same every time the experiment is conducted.

In some example embodiments, a seven-day moving average of local daily new cases (e.g., case rate) is used as the average number of newly introduced cases each day in the facility, which are the first-generation cases 710. In some cases, when calculating the seven-day average, the most recent two days are disregarded, hence the average is taken from −3 to −9 days.

Monte Carlo simulations are used to model the probability of different outcomes in a process that cannot easily be predicted due to the intervention of random variables. A Monte Carlo simulation performs analysis by building models of possible results by substituting a range of values—a probability distribution—for any factor that has inherent uncertainty. The simulation then calculates results many times, each time using a different set of random values from the probability functions. Depending upon the number of uncertainties and the ranges specified for them, a Monte Carlo simulation could involve thousands or tens of thousands of recalculations before it is complete. The Monte Carlo simulation produces distributions of possible outcome values.

The Monte Carlo simulation often follows the following operations: 1) define a domain of possible inputs; 2) generate inputs randomly from a probability distribution over the domain; 3) perform a deterministic computation on the inputs; and 4) aggregate the results.

For the simulation, a first random draw 716 is taken from the Secondary attack rate (SAR) distribution parameter 712, and a second random draw 718 is taken from the daily contact size distribution 714. The SAR distribution parameter 712 measures the proportion of successful transmission in the total number of contacted susceptible individuals for each infected individual. In some example embodiments, the SAR distribution parameter 712 is drawn from a beta distribution only at the beginning of each realization for each individual.

The daily contact size distribution 714 is drawn randomly each day from a geometric distribution. Each day, the first contacts among first-generation individuals create second generation cases, but the second-generation cases do not further transmit the disease in one example model. The daily contact size determines the degree of nodes by randomly drawing a number from a geometric distribution for each node in each day. In some example embodiments, it is assumed that 90% of average contacts in the closed-office layout occur in common spaces. The number of previous day's contacts are sampled randomly based on the probability that contacts change each day. This probability is set at 40% in some embodiments, but other values are also possible.

Based on the values drawn in the first random draw 716 and the second random draw 718, the second-generation cases 726 are calculated. The process is repeated for ten days to determine the total number of cases presented in the output 722.

To iterate for the following days, a third random draw 724 is taken from the second-generation cases 726 based on a probability 728 that the contacts for an individual change from day to day. The result is the number of people who remain in contact for the next day 720.

In the illustrated example, several assumptions are selected, but other embodiments may utilize different assumptions to change the way the simulations are performed. The assumptions include: the model keeps track of the first and second generations of infections; the transmission model does not separately account for different modes of transmission (e.g., direct contact, aerosol transmission), it is assumed that an infections case transmits with the same SAR each day of the duration when they come into the facility, but during the course of infection the transmissibility depends on the volume of shed viruses, which peaks around three days after infection.

Many simulations are executed in advance with multiple parameters and the results then stored in a database. This way, when the user changes parameters, the system does not have to run the simulations right away for the given parameters and the results are estimated by interpolating the results from known simulations with the same or similar parameter values. Interpolation in two dimensions is used along with a lookup table to provide outputs for different sets of employees and case rates to reduce the runtime within acceptable product time limits; and the lookup table for interpolation is created with 2000 simulations, but the number of simulations can be increased to improve accuracy.

It is noted that the occurrence of edges is stochastic and homogeneous, and the parameters used in the model are the susceptible population (S), the infected population (I), and the exposed population (E). In some example embodiments, the model outputs the cumulative incidence within a facility after n days, where n is 15 or less, intended for a short forecasting period.

The cases of the disease, or cumulative incidence n days post onset, is defined as the individuals who are working in the target facility within the modeling period and who meet at least one of the following criteria: clinical signs—Individuals who may have experienced the clinical signs as listed on the CDC website, including fever, cough, shortness of breaths, etc.; or diagnostic test—individuals who receive a positive result of RT-PCR test of SARS-CoV-2. Additionally.

The transmission chain is the process in which the virus spreads from one person to another. In the contact network model, the transmission chain is represented by series of nodes (susceptible, exposed, and infected individuals) and edges (contacts).

The first-generation cases are individuals who are the starting point of a transmission chain. These include one or more index cases that are infected outside a facility and introduce the disease into the facility. This group is considered as the infected population (I).

The second-generation cases are individuals who are exposed to COVID-19 as a result of a successful transmission event after coming into contact with the first-generation cases. This group forms the exposed population (E). In the present model, it is assumed that these individuals do not become infectious within the short forecasting period. Hence, the transmission chain ends at the second-generation cases.

The onset of COVID-19 cases in a facility is defined as the time when the first case arrived in the facility, and it does not refer to onset of symptoms. Further, a contact is defined as one of the following events, that could lead to the exposure of COVID-19 in a susceptible population, taking place: a face-to-face interaction with a case within the facility for at least 15 minutes: direct physical contact with a case within the facility; or other type of situations that could lead to the exposure to infectious material, for example, working with a case in the same room, or touching the same surface.

The following pseudo code illustrates the design of the model, according to some example environments:

| | |
|---|---|
| p_{case} | // daily new case rate |
| e | // total employees or nodes |
| n_{days} | // total number of modeling days |
| α_{SAR} | // alpha parameter for SAR Beta distribution |
| β_{SAR} | // beta parameter for SAR Beta distribution |
| c | // average daily contact size |
| pᵣₑₘₐᵢₙ | // probability that edges (contacts) are sustained for the next day |
| nₛᵢₘ | // number of simulations |

```
for all sim ∈ nₛᵢₘ do
  Sample number of first-generation cases for n_{days},
    n_{g1} := (g1⁽¹⁾, . . . , g1^{(ndays)}) ~ Binomial(e, p_{case})
  Initialize empty array of node indices for first-generation cases, g1:=( )
  for all g1 ∈ Σ n_{g1} do
    Sample SAR for each first-generation case, SAR_{g1} ~ Beta(α_{SAR},
    β_{SAR})
    Initialize empty array of previous day's contacts for each first-
      generation case, c_remain_{g1} := ( )
  end for
  for all d ∈ n_{days} do
    Sample unique indices of first-generation cases for day
      g1=g1 + Uniform(e, g1^{(d)})
    for all g1 ∈ g1 do
      Sample nu̅m̅ber of contacts for each first-generation case for
        day n_{cg1} ~ Geometric (1/(c+1))
      Sample indices of new contacts, c_new_{g1} ~ Uniform(e, n_{cg1} −
        size(c_remaing₁))
      Total secondary contacts for day, c_{g1} := c_new_{g1} +
        c_remain_{g1}
      Sample indices of second-generation cases from contacts for
        day, g2^{(d)} ~ Binomial(c_{g1,} SAR_{g1})
      Sample contacts that remain consistent the next day,
      c_remain_{g1} ~ Binomial(^{c}g1, Premain)
    end for
  end for
Total cumulative incidence, casesₛᵢₘ := Unique(g1 + Σ_{d} g2^{(d)})
end for
```

The first-generation cases who are infected from the community can arrive at the facility on any given day. It is assumed that these cases are introduced to the facility as Bernoulli trials using local daily new case rate as the success rate.

Once first-generation cases arrive, random contacts are initiated between the infected and the susceptible individuals. The daily contact size of the first-generation cases is randomly determined from a modified geometric distribution based on the average daily contacts. Further, the employees contacted by the first-generation case(s) are randomly chosen from the population. Additionally, the model has a parameter to probabilistically determine how many contacts remain as contacts for the next day.

The transition from susceptible to exposed state takes place among the employees who make contacts with the first-generation cases based on the secondary attack rate (SAR). A randomized SAR drawn from a Beta distribution is assigned to each first-generation case for the entire modeling period. The average SAR for a workplace is determined based on mask compliance, airflow, filtration, speaking volume, and speaking percentage.

Given the short forecasting period of the model, we do not track third-generation cases. Therefore, employees who are infected by first-generation cases remain in the exposed state and do not further transmit during the modeling period.

It is assumed that the first-generation cases are introduced to a facility as Bernoulli trials using local daily new case rate as the success rate. Therefore, the probability P(x) of x infected employees arriving at a facility on any given day is calculated from a Binomial distribution as follows:

$$P ( x ) = Binomial ( x , e , {p}_{case} ) == \frac{e !}{x ! ( e - x ) !} {{p}_{case}^{x} ( 1 - {p}_{case} )}^{e - x}$$

Here, e is the total number of employees, and p_{case} is the local daily new case rate. The assumption of Binomial distribution matches similar approaches used in other studies for modeling probability of case occurrence.

The Secondary Attack Rate (SAR) is defined as the probability of an infection being transferred to a naive individual. In some example embodiments, it is approximated as the proportion of secondary cases induced in a contact group by the first case in the group. In the present model, SAR is the probability of getting exposed among all secondary contacts of a first-generation case. In other words, SAR is the probability of getting exposed among all secondary contacts of a first-generation case.

Multiple studies on COVID-19 outbreaks have reported a wide range of SAR across different settings. High values of SAR have been observed in specific settings, such as a SAR of 53.3% during loud singing, a SAR of 84.6% after a business meeting, and a SAR of 38.8% for people having a meal together. Meta-analysis of SAR for COVID-19 concluded that workplaces where close contacts are less intense and frequent tend to have lower values of SAR (0%-5.3%) than household settings (3.9%-54.9%).

The SAR associated with each infected individual varies because of their viral load and course of infection. This variability is incorporated into the model by sampling SAR for each first-generation case from the Beta distribution. The SAR assigned to each first-generation case remains constant throughout the forecast period as a simplifying assumption. The Beta distribution is selected because it is defined on the desired interval [0, 1] and is able to model long tails. The distribution has two parameters: α_{SAR} and β_{SAR}. The probability distribution function for the Beta distribution, ƒ(SAR=x) is defined as—

$$f ( S A R = x ) = Beta ( x , {α}_{S A R} , {β}_{S A R} ) = ( Γ ( {α}_{S A R} + {β}_{S A R} ) / Γ ( {α}_{S A R} ) Γ ( {β}_{S A R} ) ) \cdot {{x}^{{α}_{S A R} - 1} ( 1 - x )}^{{β}_{S A R} - 1}$$

Here, Γ( ) is the gamma function. It is expected that the SAR distribution in a population is long tailed, where a majority of infected individuals exhibit low SAR or infect few people, while a minority of individuals exhibit high SAR. A long-tail distribution can be implemented in the Beta distribution by setting α_{SAR}=1. Then, the β_{SAR} is calculated from the average SAR across multiple individuals. The Beta distribution corresponding is defined as:

β_{SAR}=α_{SAR}(1−S̅A̅R̅)/S̅A̅R̅

Studies have concluded that SAR associated with the transmission of airborne pathogens vary based on particle emission and inhalation rates, airflow, and filtration. In some example embodiments, particle emission and inhalation are determined based on speaking percentage, speaking volume, and mask effectiveness.

The SAR for airborne transmission of COVID-19 is described as

SAR=1−exp^{-N/N0}

N is the total number of virions breathed in, while N₀ is the threshold of virions required to infect a susceptible individual, or the infectivity threshold. Further, the number of inhaled virions N is estimated as follows:

$$N = B T {C}_{e q} ( 1 - \frac{1}{λ T} ( 1 - {exp}^{- λ T} ) )$$

$$and$$

$${C}_{e q} = S / λ V$$

Here, B is breathing rate, T is time exposed, C_{eq} is the concentration of virions in a steady state, λ is the decay rate of virions, S is the virion emission at the source, and V is the volume of the room.

In some example embodiment, one or more some simplifying assumptions are used from the following:
It is assumed independent transmission on each day from an infectious individual to susceptible contacts, hence T=1 and is constant.
The term 1−(1/λT)(1−exp^{-λT}) accounts for virion decay over short periods of time. For transmission over longer duration like a day, the term may be ignored.
It is assumed that the breathing rate B is constant. The breathing rate changes with activities like heavy breathing when exercising, which is not expected to occur commonly within a workplace. Breathing rate also changes based on whether masks are worn, however we have combined the contribution of masks from both source emission rate and breathing rate into a single additional parameter mₑ for simplification.
For a general facility, the volume (V) is ignored, since in most cases, COVID-19 is only expected to be transmitted within the vicinity of an infected individual, for example, within 6 ft, hence, the room volume would not contribute significantly to changes in SAR.
While the infectivity threshold varies by individual, for the assessment of average SAR, the average infectivity threshold, No is assumed as constant. For simplification, N₀ is replaced with N^{t} in the equations below. Then, the equation is simplified as follows:

$${N}^{′} ∝ \frac{S ( 1 - {m}_{e} )}{λ}$$

$$⇒ \frac{{N}_{i}^{′}}{{N}_{j}^{′}} = \frac{{S}_{i} ( 1 - {m}_{{e}_{i}} )}{{λ}_{i}} \frac{{λ}_{j}}{{S}_{j} ( 1 - {m}_{{e}_{j}} )}$$

Additionally:

SAR=1−exp(−N′)

Subscripts i and j represent any two sets of parameters, and mₑ [0, 1] is the mask effectiveness. The source emission rate S is a function of speaking percentage and volume. The decay rate λ is a function of airflow and filtration.

In some example embodiments, it is assumed that the source emission rate S is a function of speaking percentage sₚ, and speaking volume sᵥ in decibels. It was observed from case reports that talking releases 46 times more virions than breathing. Other studies have concluded that higher volume releases higher number of aerosolized particles carrying virions. The increase in virions is represented based on volume by the volume multiplier sₘ. Then, the source emission S is described as:

S=(1−sₚ)+46sₚsₘ

Based on measurements from 10 participants, increasing the speaking volume from 70 dB to 98 dB increased the particle emission rate from 6 to 53 particles per second. Additionally, the particle emission rate is directly proportional to the root mean square amplitude of the vocalization. Since the decibel level depends on the logarithm of amplitude, the relationship with emitted particles or virions q can be described as—

sᵥ∝log(q)

⇒sᵥ=C_{q} log(q)

C_{q} is an unknown constant is computed as follows:

C_{q}=(98−70)/log(53/6)

Assuming that the ratio of 46 virions corresponds to speaking at a reference volume of 60 dB, the activity multiplier sₘ at an arbitrary volume level can then be computed as follows:

$${s}_{m} = \frac{{q}_{{s}_{v}}}{{q}_{60}}$$

$$= exp ( \frac{{s}_{v} - 60}{{C}_{q}} )$$

$$= {\frac{53}{6}}^{( {s}_{v} - 60 ) / ( 98 - 70 )}$$

The decay rate λ is a function of air exchange rate or airflow λₐ (air changes per hour or ACH), viral settling and deactivation λ_{d}=0.62/h, and decay due to filtration λ_{ƒ}. The decay rate λ is calculated as follows:

λ=λₐ+λ_{d}+λ_{ƒ}

Based on a meta-analysis on the impact of mask-wearing on COVID-19 transmission, the relative risk (RR) of mask wearing under non-healthcare setting is RR=56%, and for healthcare setting is RR=30%. Then, mask effectiveness can be implemented in equation as the factor mₑ=1−RR.

The equations presented above are combined to estimate the SAR for any set of speaking percentage, volume, airflow, filtration, and mask effectiveness, given a reference SAR and its corresponding parameters.

Different studies have presented SAR among non-household contacts in the range of 0%-5.3%. In some example embodiments, a reference SAR, SAR_{ref} of 5.1% is used as the one associated with non-household unprotected contacts from the Germany outbreak study in a workplace setting, and the following associated parameters are assumed: airflow λₐ=2 ACH; filtration λ_{ƒ}=0, speaking percentage sₚ=25%, speaking volume sᵥ=65 dB, and mask effectiveness mₑ=0.

Daily contact size is defined as the number of secondary contacts of each first-generation case on each day, and is randomly sampled from a modified Geometric distribution. Given the average daily contacts c̅, the probability of daily contacts P(c=x) is defined as follows:

$$P ( c = x ) = Geometric ( \frac{1}{\overline{c} + 1} )$$

$$= {( 1 - \frac{1}{\overline{c} + 1} )}^{x} ( \frac{1}{\overline{c} + 1} )$$

The parameterization of the Geometric distribution is based on data collected by the citizen science based a BBC pandemic study. The workplace contacts in the BBC study align well with data from an independent POLYMOD study. Based on the BBC study, the average daily contacts at work are c=6.

To obtain a range around the mean for validation purposes, the data is transformed so follows the Gaussian distribution. It was observed that there was a considerable number of observations with zero daily contacts (up to 35th percentile).

Additionally, it is expected that employees contact some of the same people every day at work. The model incorporated this using a parameter for the probability that contacts remain the same the next day pᵣₑₘₐᵢₙ. It is assumed that pᵣₑₘₐᵢₙ=60% based on an office study in Italy.

The contact network model presented above can be effectively used to assess return-to-work policies in workplaces. Some examples of control measures include the following:
Work from home—When a percentage of employees work remotely, it reduces the number of employees e in the workplace. This reduces the number of case introductions, and hence cumulative incidence post onset.
Mask wearing: Masks reduce both the source emission and the breathing rate for virions, and is implemented in the model using the parameter mₑ. Wearing masks reduces the number of virions N^{t}, thus reducing average SAR, and hence cumulative incidence.
Ventilation improvement: increasing airflow and incorporating filtration increases the decay rate λ, resulting in reduction in average SAR.
Vocal loudness and speaking percentage: decreasing vocal loudness and the amount of talking leads to lower amounts of emitted virions S. This in turn reduces average SAR.
Social distancing: social distancing reduces the average daily contacts c in the model, thus reducing cumulative incidence.

The following is a summary of the key modeling and parameter assumptions:

Assumptions for Transmission Dynamics
No differentiation among multiple modes of transmission, such as airborne, droplet, and direct contact.
The length of the incubation period is not considered. Studies have suggested a median exposure to infectious period of approximately 5 days. Given the objective of a short-term forecasts with n_{days}≤15, it is assumed that the second-generation cases do not further transmit the disease within this period. In other words, it is assumed that the first-generation cases remain in the infectious state, while the second-generation cases remain in the exposed state after a successful transmission event.
No additional mitigation and intervention measures are considered in the middle of the modeling period. For example, it is assumed that no individuals, infected or otherwise, are removed from the facility.

Assumptions for Model Parameters
First-generation cases are sampled using Bernoulli trials for each employee in the facility with the probability of success as the local daily new case rate.
SAR has a significant level of heterogeneity across different conditions such as type of contacts, age, sex, presence of symptoms, and environment. The present model has considered the heterogeneity based on mask effectiveness, airflow, speaking volume, and speaking percentage. Other host factors that lead to the heterogeneity such as age, course of the infection, and physiological condition of infected individuals are not considered.
SAR is randomly sampled for each first-generation case from a Beta distribution determined by the average SAR that is dependent on the environmental characteristics and employee behavior within a facility.
The randomly sampled SAR assigned to each first-generation case remains constant throughout the modeling period. In this way, the sampled SAR for each individual may be considered as the average temporal SAR, since in reality, the SAR would change over time based on the dynamics of viral load during different courses of a disease.
The number of daily contacts for each individual each day is determined by a Geometric distribution. The average contacts for the Geometric distribution are based on self-reported data from the BBC pandemic study.

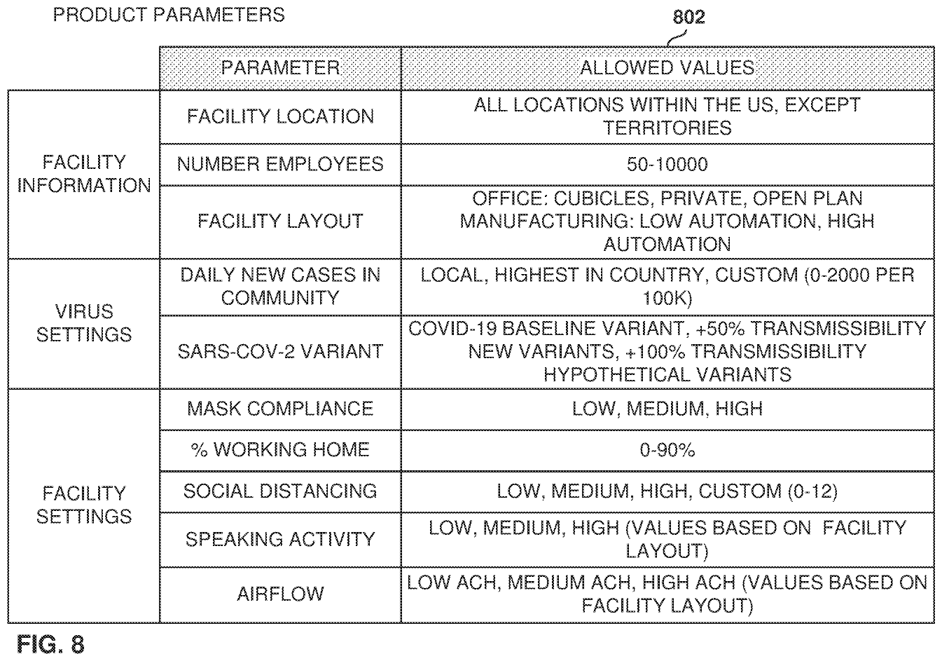

FIG. 8 is a table 802 with product parameters and the allowed values, according to some example embodiments. The parameters configurable by the user may be adjusted according to the situation. FIGS. 8-10 illustrate some of the parameters and the possible ranges.

The parameters in Table 802 can be from several categories: facility information, virus settings and facility settings. The facility information includes several parameters for the facility, including facility location, number of employees, and facility layout. The virus settings include daily new cases in the community and variant information. Further, the facility settings include mask compliance, percentage of people working from home, social distancing, speaking activity, and airflow.

In some example embodiments, the facility location is a location within the United States, but other countries are also possible. The number of employees for the facility has a value between 50 and 10,000, but other ranges are also possible, e.g., 10-20,000. Further, the facility layout may be cubicles, private, and open plan for office space, and low or high automation for manufacturing facilities.

The daily new cases in the community may be set to local, highest in the country, or custom, which may be set to a value between 0 to 2000 per hundred thousand population. The SARS-CoV-2 variant may be set to one of COVID-19 Baseline Variant, higher than 50% Transmissibility New Variants, and higher than 100% Transmissibility Hypothetical Variants.

The mask compliance may be set to low, medium or high. The work-from-home rates is a value between zero and 90%. The social-distance-compliance rates is one of low, medium, high, or custom (e.g., an integer value between 0 and 12). The speaking activity may be one of low, medium, or high, and the values depend on the facility layout. The speaking activity may be set to low ACH, medium ACH, or high ACH, and the values depend on the facility layout.

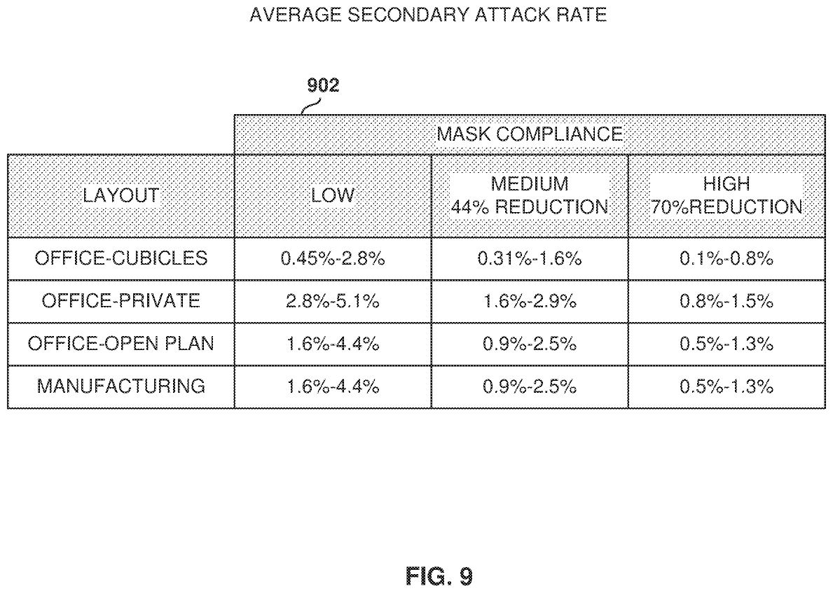

FIG. 9 is a table 902 with allowed values based on mask compliance and building layout, according to some example embodiments. The secondary attack rate is the probability that successful transmission occurs among susceptible individuals following known contact with an infectious individual or an infectious source within a reasonable incubation period.

The activity level and airflow based on layout is taken into consideration, and the individual SAR is a sample from the beta distribution, as discussed above.

As illustrated in table 902, the secondary attack rate is based on the layout (e.g., office cubicles, private offices, open-plan office, and manufacturing) and the mask compliance, which can be low, medium, or high. Based on these two factors, the ranges for each combination is set, e.g., for low mask compliance in a cubicle layout, the secondary attack rate is between 0.45% and 2.8%.

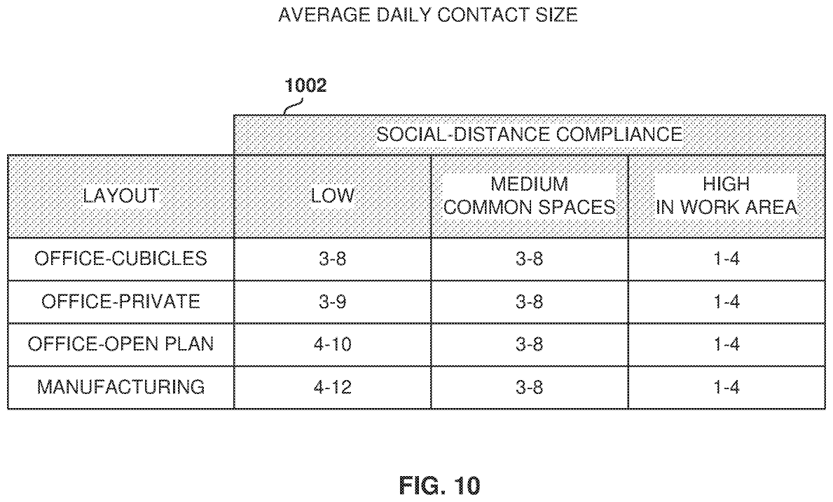

FIG. 10 is a table 1002 with allowed values based on social-distance compliance and building layout, according to some example embodiments. In some example embodiments, average daily contacts is based on a BBC pandemic study from 2018. In this study, people were asked if they contacted someone or not, but there is no data to differentiate between close contact (e.g., 1 ft), or distanced contact (e.g., six feet).

Table 1002 shows the average daily contact size based on the office layout and the social distancing compliance, which could be low medium or high. For example, for medium socio-distance compliance in a private office set up, the average daily contact is in the range from 3 to 8.

It is noted that the embodiments illustrated in FIGS. 8-10 are examples and do not describe every possible embodiment. Other embodiments may utilize different parameters, different ranges, additional bin values, etc. The embodiments illustrated in FIGS. 8-10 should therefore not be interpreted to be exclusive or limiting, but rather illustrative.

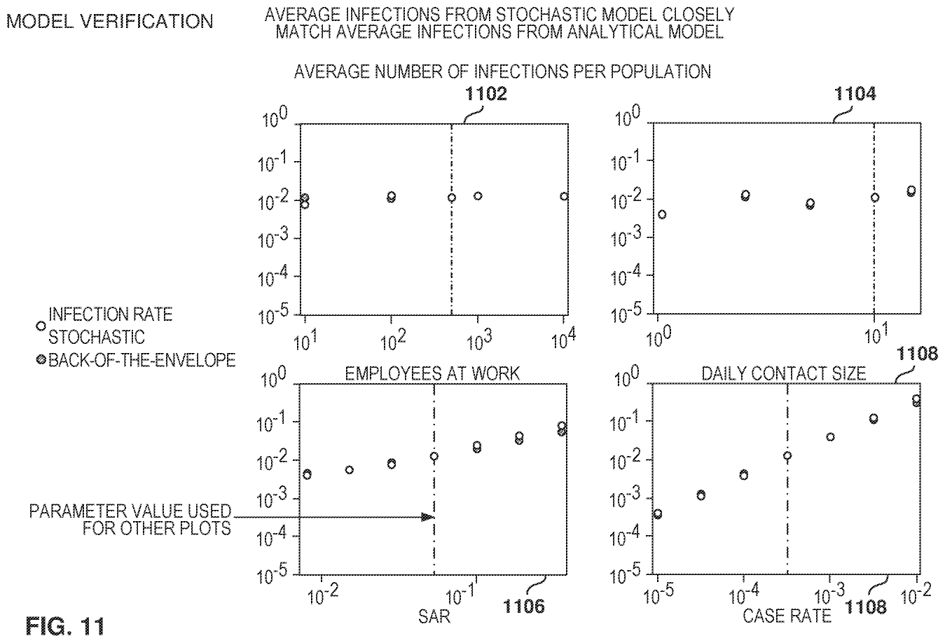

FIG. 11 illustrates some of the metrics for verifying the model, according to some example embodiments. Some of the challenges to verify the validity of the model is that it is difficult to obtain disease-transmission data, as companies are reluctant to share this type of information.

In one case validation was performed against known data gathered from nursing homes. The data for 8300 nursing homes associated with Medicare and Medicaid was used and compared to the simulations. Charts 1102-1108 show the average number of infections for the number of employees at work, daily contact size, SAR, and case rate. The plots show a comparison of the plotted values for the model versus the actual data.

Charts 1102 and 1106 are for the nursing-home data, and charts 1104 and 1108 are for the model estimates. The results for both cases are close, which shows the validity of the models. This example is for illustration purposes, and other testing may provide different values.

The model may be used for COVID-19 or for other infectious diseases, and the corresponding parameters are adjusted based on experience. For example, the model may be used for influenza, the common cold, etc. Also, when new strains appear, the strains are studied and parameters adjusted accordingly. For example, a new strain may prove to be more contagious, so the model will increase the parameters associated with infecting from one individual to another.

Further, the model may be adjusted for other types of gathering places, such as churches, sporting events (indoors and outdoors), airplanes, schools, etc.

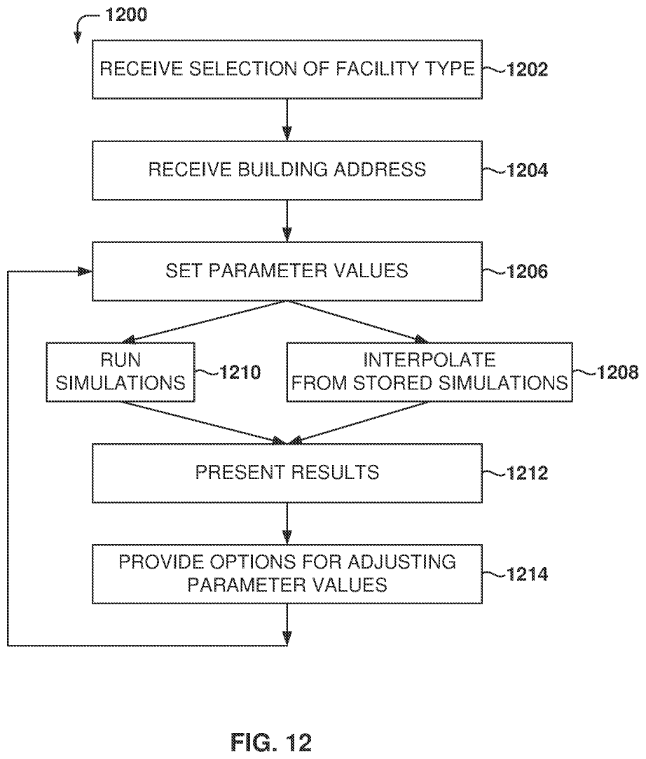

FIG. 12 is a flowchart of a method 1200 for interacting with the planning tool, according to some example embodiments. While the various operations in this flowchart are presented and described sequentially, one of ordinary skill will appreciate that some or all of the operations may be executed in a different order, be combined or omitted, or be executed in parallel.

The UI for the planning tool is presented to the user, such as the UI of FIGS. 1 and 2. At operation 1202, the selection of facility is received, e.g., office with cubicles.

From operation 1202, the method 1200 flows to operation 1204 where, after presenting the option to enter the building address (e.g., as shown in FIG. 3), the building address is received. In some cases, a check is made to determine if the building address is an existing building address, and if it is not, the user will be asked to enter the correct address.

At operation 1206, the parameters for the simulation to estimate the infection spread are set. For example, by selecting the corresponding parameters from the tables of FIGS. 8-10.

Once the parameter values are set, the method 1200 flows to either operation 1210 or to operation 1208. If there is time to run simulations, operation 1210 is executed and the simulation is run, e.g., as illustrated in FIG. 7. If a quick response is desired, operation 1208 is performed to interpolate the results from previously run simulations accordingly to the set parameter values.

At operation 1212, the results are presented in the UI, either from running the simulation or from the interpolation. The UI, at operation 1214, provides options for adjusting the parameter values so the user can find the results and change different scenarios for the facilities, such as changing the social distance or the number of employees working from home.

If the user changes the parameters, the method 1200 flows back to operation 1206, and the process is repeated to obtain new results and present them to the user in the UI.

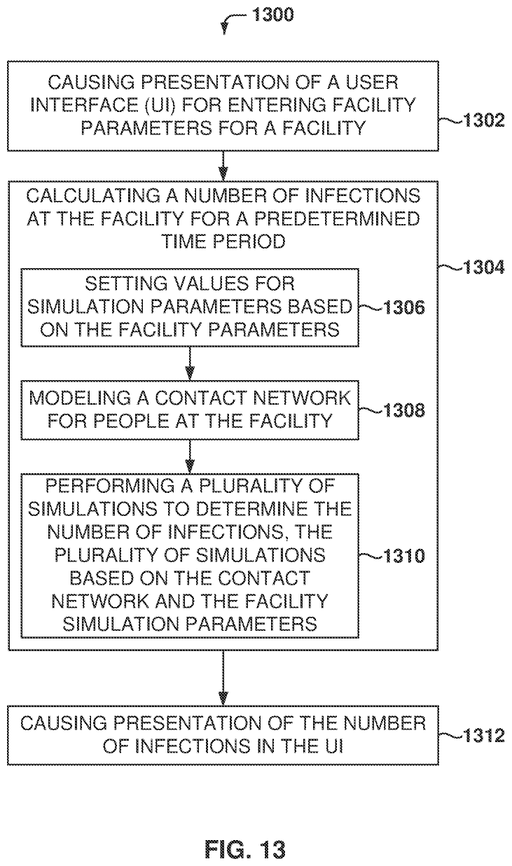

FIG. 13 is a flowchart of a method 1300 for estimating disease-spreading based on facility and behavioral parameters, according to some example embodiments.

Operation 1302 is for causing presentation of the UI for entering facility parameters for a facility.

From operation 1302, the method 1300 flows to operation 1304 for calculating a number of infections at the facility for a predetermined time period. The operation 1304 includes operations 1306, 1308, and 1310.

At operation 1306, values for simulation parameters are set based on the facility parameters, and operation 1308 is for modeling a contact network for people at the facility. At operation 1310, a plurality of simulations are performed to determine the number of infections, the plurality of simulations being based on the contact network and the facility simulation parameters

From operation 1304, the method 1300 flows to operation 1312 for causing presentation of the number of infections in the UI.

In some embodiments, the facility parameters comprise office facility and manufacturing facility; the office facility comprising open-floor plan, cubicles, and private offices; and the manufacturing facility comprising low automation and high automation.

In some embodiments, the simulation parameters include one or more of a local infection case rate, a mask-compliance parameter, a percentage of workers working from home, and a social-distancing compliance parameter.

In some embodiments, each simulation comprises drawing a first value from a disease transmission distribution, drawing a second value from a daily contact size distribution, and determining a number of next generation cases based on the first value and the second value.

In some embodiments, causing presentation of the number of infections in the UI comprises an expected range of infections in the facility and a maximum number of infections in the facility.

In some embodiments, the contact network models transmission of the disease caused by social contact for an airborne infection, wherein two individuals are linked in the contact network if there is sufficient contact to allow the infection to pass between the two individuals.

In some embodiments, causing presentation of the number of infections in the UI comprises providing options in UI for configuring simultaneously a plurality of scenarios for calculating infections.

In some embodiments, the method 1300 further comprises providing allowed values for a plurality of parameters comprising facility location, number of employees, facility layout, local case rate, percentage of people working from home, social-distance compliance, and mask-wearing compliance.

In some embodiments, the method 1300 further comprises providing ranges of values for a secondary attack rate for transmission based on the facility layout and the mask-wearing compliance.

In some embodiments, the method 1300 further comprises providing ranges of values for an average daily contact size based on the social-distance compliance and the facility layout.

Another general aspect is for a system that includes a memory comprising instructions and one or more computer processors. The instructions, when executed by the one or more computer processors, cause the one or more computer processors to perform operations comprising: causing presentation of a user interface (UI) for entering facility parameters for a facility, calculating a number of infections at the facility for a predetermined time period, the calculating the number of infections further comprising: setting values for simulation parameters based on the facility parameters; modeling a contact network for people at the facility; and performing a plurality of simulations to determine the number of infections, the plurality of simulations based on the contact network and the facility simulation parameters; and causing presentation of the number of infections in the UI.

In yet another general aspect, a machine-readable storage medium (e.g., a non-transitory storage medium) includes instructions that, when executed by a machine, cause the machine to perform operations comprising: causing presentation of a user interface (UI) for entering facility parameters for a facility; calculating a number of infections at the facility for a predetermined time period, the calculating the number of infections further comprising: setting values for simulation parameters based on the facility parameters; modeling a contact network for people at the facility; and performing a plurality of simulations to determine the number of infections, the plurality of simulations based on the contact network and the facility simulation parameters; and causing presentation of the number of infections in the UI.

In view of the disclosure above, various examples are set forth below. It should be noted that one or more features of an example, taken in isolation or combination, should be considered within the disclosure of this application.

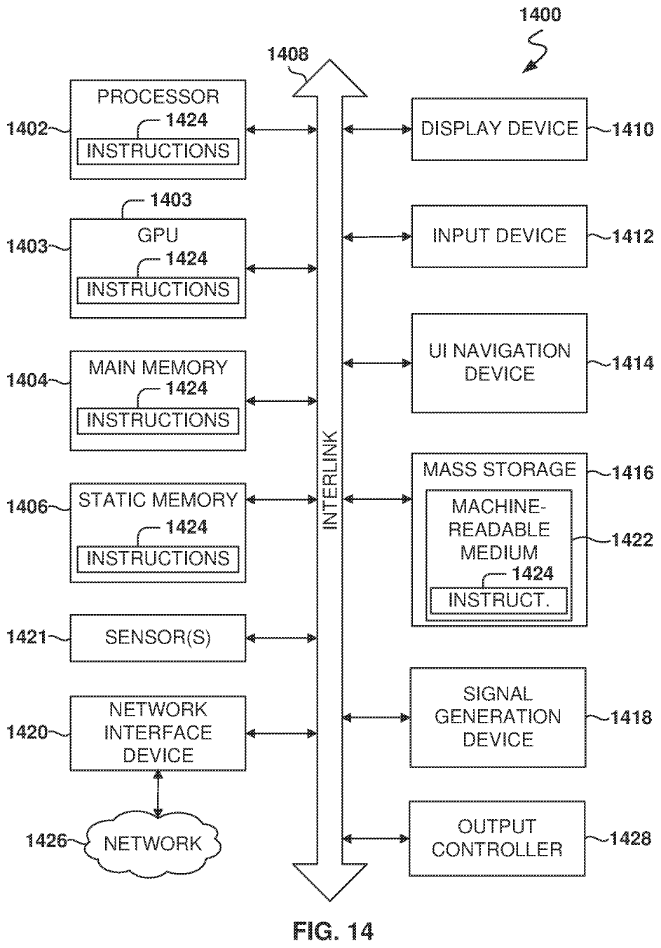

FIG. 14 is a block diagram illustrating an example of a machine 1400 upon or by which one or more example process embodiments described herein may be implemented or controlled. In alternative embodiments, the machine 1400 may operate as a standalone device or may be connected (e.g., networked) to other machines. In a networked deployment, the machine 1400 may operate in the capacity of a server machine, a client machine, or both in server-client network environments. In an example, the machine 1400 may act as a peer machine in a peer-to-peer (P2P) (or other distributed) network environment. Further, while only a single machine 1400 is illustrated, the term “machine” shall also be taken to include any collection of machines that individually or jointly execute a set (or multiple sets) of instructions to perform any one or more of the methodologies discussed herein, such as via cloud computing, software as a service (SaaS), or other computer cluster configurations.

Examples, as described herein, may include, or may operate by, logic, a number of components, or mechanisms. Circuitry is a collection of circuits implemented in tangible entities that include hardware (e.g., simple circuits, gates, logic). Circuitry membership may be flexible over time and underlying hardware variability. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits) including a computer-readable medium physically modified (e.g., magnetically, electrically, by moveable placement of invariant massed particles) to encode instructions of the specific operation. In connecting the physical components, the underlying electrical properties of a hardware constituent are changed (for example, from an insulator to a conductor or vice versa). The instructions enable embedded hardware (e.g., the execution units or a loading mechanism) to create members of the circuitry in hardware via the variable connections to carry out portions of the specific operation when in operation. Accordingly, the computer-readable medium is communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry, at a different time.

The machine (e.g., computer system) 1400 may include a hardware processor 1402 (e.g., a central processing unit (CPU), a hardware processor core, or any combination thereof), a graphics processing unit (GPU) 1403, a main memory 1404, and a static memory 1406, some or all of which may communicate with each other via an interlink (e.g., bus) 1408. The machine 1400 may further include a display device 1410, an alphanumeric input device 1412 (e.g., a keyboard), and a user interface (UI) navigation device 1414 (e.g., a mouse). In an example, the display device 1410, alphanumeric input device 1412, and UI navigation device 1414 may be a touch screen display. The machine 1400 may additionally include a mass storage device (e.g., drive unit) 1416, a signal generation device 1418 (e.g., a speaker), a network interface device 1420, and one or more sensors 1421, such as a Global Positioning System (GPS) sensor, compass, accelerometer, or another sensor. The machine 1400 may include an output controller 1428, such as a serial (e.g., universal serial bus (USB)), parallel, or other wired or wireless (e.g., infrared (IR), near field communication (NFC)) connection to communicate with or control one or more peripheral devices (e.g., a printer, card reader).

The mass storage device 1416 may include a machine-readable medium 1422 on which is stored one or more sets of data structures or instructions 1424 (e.g., software) embodying or utilized by any one or more of the techniques or functions described herein. The instructions 1424 may also reside, completely or at least partially, within the main memory 1404, within the static memory 1406, within the hardware processor 1402, or within the GPU 1403 during execution thereof by the machine 1400. In an example, one or any combination of the hardware processor 1402, the GPU 1403, the main memory 1404, the static memory 1406, or the mass storage device 1416 may constitute machine-readable media.

While the machine-readable medium 1422 is illustrated as a single medium, the term “machine-readable medium” may include a single medium, or multiple media, (e.g., a centralized or distributed database, and/or associated caches and servers) configured to store the one or more instructions 1424.

The term “machine-readable medium” may include any medium that is capable of storing, encoding, or carrying instructions 1424 for execution by the machine 1400 and that cause the machine 1400 to perform any one or more of the techniques of the present disclosure, or that is capable of storing, encoding, or carrying data structures used by or associated with such instructions 1424. Non-limiting machine-readable medium examples may include solid-state memories, and optical and magnetic media. In an example, a massed machine-readable medium comprises a machine-readable medium 1422 with a plurality of particles having invariant (e.g., rest) mass. Accordingly, massed machine-readable media are not transitory propagating signals. Specific examples of massed machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1424 may further be transmitted or received over a communications network 1426 using a transmission medium via the network interface device 1420.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

As used herein, the term “or” may be construed in either an inclusive or exclusive sense. Moreover, plural instances may be provided for resources, operations, or structures described herein as a single instance. Additionally, boundaries between various resources, operations, modules, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

What is claimed is:

1. A computer-implemented method comprising:
causing, by one or more processors, presentation of a user interface (UI) for entering facility parameters for a facility;
calculating, by the one or more processors, a number of infections at the facility for a predetermined time period, the calculating the number of infections further comprising:
setting values for simulation parameters based on the facility parameters;
modeling a contact network for people at the facility; and
performing a plurality of simulations to determine the number of infections, the plurality of simulations based on the contact network and the facility simulation parameters; and
causing, by the one or more processors, presentation of the number of infections in the UI.

2. The method as recited in claim 1, wherein the facility parameters comprise office facility and manufacturing facility; the office facility comprising open-floor plan, cubicles, and private offices; and the manufacturing facility comprising low automation and high automation.

3. The method as recited in claim 1, wherein the simulation parameters include one or more of a local infection case rate, a mask-compliance parameter, a percentage of workers working from home, and a social-distancing compliance parameter.

4. The method as recited in claim 1, wherein each simulation comprises:
drawing a first value from a disease transmission distribution;
drawing a second value from a daily contact size distribution; and
determining a number of next generation cases based on the first value and the second value.

5. The method as recited in claim 1, wherein causing presentation of the number of infections in the UI comprises:
an expected range of infections in the facility and a maximum number of infections in the facility.

6. The method as recited in claim 1, wherein the contact network models transmission of a disease caused by social contact for an airborne infection, wherein two individuals are linked in the contact network if there is sufficient contact to allow the infection to pass between the two individuals.

7. The method as recited in claim 1, wherein causing presentation of the number of infections in the UI comprises:
providing options in the UI for configuring simultaneously a plurality of scenarios for calculating infections.

8. The method as recited in claim 1, further comprising:
providing allowed values for a plurality of parameters comprising facility location, number of employees, facility layout, local case rate, percentage of people working from home, social-distance compliance, and mask-wearing compliance.

9. The method as recited in claim 8, further comprising:
providing ranges of values for a secondary attack rate for transmission based on the facility layout and the mask-wearing compliance.

10. The method as recited in claim 8, further comprising:
providing ranges of values for an average daily contact size based on the social-distance compliance and the facility layout.

11. A system comprising:
a memory comprising instructions; and
one or more computer processors, wherein the instructions, when executed by the one or more computer processors, cause the system to perform operations comprising:
causing presentation of a user interface (UI) for entering facility parameters for a facility;
calculating a number of infections at the facility for a predetermined time period, the calculating the number of infections further comprising:
setting values for simulation parameters based on the facility parameters;
modeling a contact network for people at the facility; and
performing a plurality of simulations to determine the number of infections, the plurality of simulations based on the contact network and the facility simulation parameters; and
causing presentation of the number of infections in the UI.

12. The system as recited in claim 11, wherein the facility parameters comprise office facility and manufacturing facility, the office facility comprising open-floor plan, cubicles, and private offices; and the manufacturing facility comprising low automation and high automation.

13. The system as recited in claim 11, wherein the simulation parameters include one or more of a local infection case rate, a mask-compliance parameter, a percentage of workers working from home, and a social-distancing compliance parameter.

14. The system as recited in claim 11, wherein each simulation comprises:
drawing a first value from a disease transmission distribution;
drawing a second value from a daily contact size distribution; and
determining a number of next generation cases based on the first value and the second value.

15. The system as recited in claim 11, wherein causing presentation of the number of infections in the UI comprises:
an expected range of infections in the facility and a maximum number of infections in the facility.

16. A tangible machine-readable storage medium including instructions that, when executed by a machine, cause the machine to perform operations comprising:
causing presentation of a user interface (UI) for entering facility parameters for a facility;
calculating a number of infections at the facility for a predetermined time period, the calculating the number of infections further comprising:
setting values for simulation parameters based on the facility parameters;
modeling a contact network for people at the facility; and
performing a plurality of simulations to determine the number of infections, the plurality of simulations based on the contact network and the facility simulation parameters; and
causing presentation of the number of infections in the UI.

17. The tangible machine-readable storage medium as recited in claim 16, wherein the facility parameters comprise office facility and manufacturing facility; the office facility comprising open-floor plan, cubicles, and private offices; and the manufacturing facility comprising low automation and high automation.

18. The tangible machine-readable storage medium as recited in claim 16, wherein the simulation parameters include one or more of a local infection case rate, a mask-compliance parameter, a percentage of workers working from home, and a social-distancing compliance parameter.

19. The tangible machine-readable storage medium as recited in claim 16, wherein each simulation comprises:
drawing a first value from a disease transmission distribution;
drawing a second value from a daily contact size distribution; and
determining a number of next generation cases based on the first value and the second value.

20. The tangible machine-readable storage medium as recited in claim 16, wherein causing presentation of the number of infections in the UI comprises:
an expected range of infections in the facility and a maximum number of infections in the facility.
